(19) United States
(12) Patent Application Publication
(10) Pub. No.: US 2025/0288244 A1
(43) Pub. Date: Sep. 18, 2025

(54) ELECTRONIC DEVICE, CONTROL METHOD, AND COMPUTER PROGRAM FOR EVALUATING AND ANALYZING SPIRAL DRAWING FOR DIAGNOSTIC MEASUREMENT OF MOVEMENT ABILITY

(71) Applicant: BEAUBRAIN HEALTHCARE INC., Seoul (KR)

(72) Inventors: Ji Hye YOON, Seoul (KR); Duk Lyul NA, Seoul (KR); Jin Young YOUN, Seoul (KR)

(73) Assignee: BEAUBRAIN HEALTHCARE INC., Seoul (KR)

(21) Appl. No.: 19/123,749

(22) PCT Filed: Dec. 23, 2022
(86) PCT No.: PCT/KR2022/021240

§ 371 Date: Apr. 23, 2025

(30) Foreign Application Priority Data
Jul. 12, 2022 (KR) 10-2022-0085472

(51) Int. Cl. A61B 5/00

(52) U.S. Cl. CPC A61B 5/4082; A61B 5/7271; A61B 5/742; A61B 5/7475

(57) ABSTRACT

According to an aspect of the present disclosure, an electronic device for evaluating and analyzing spiral drawing for diagnostic measurement of movement ability: tracks a line produced by writing pressure to identify at least one stroke constituting the line; calculates the length of the stroke; calculates the writing pressure applied on a display with respect to the stroke; calculates the time taken to produce the stroke; and calculates the speed on the basis of the length of the stroke and the time taken.

### TECHNICAL FIELD

The present disclosure relates to an electronic device, a control method, and a computer program for evaluating and analyzing spiral drawing for diagnostic measurement of movement ability, and more particularly, to a technology for diagnosing whether a user's movement ability requires improvement based on writing pressure and line images acquired by requesting line drawing according to a proposed drawing method.

### BACKGROUND ART

Conventionally, diagnosis of movement ability has been conducted based on sensing values measured through sensors attached to the body, but this requires expensive equipment and diagnosis is only performed in limited spaces, which has led to problems in which patients with movement disorders or the general public who wish to have their movement ability diagnosed cannot receive a diagnosis due to these limitations.

Movement disorder refers to a disability that makes it impossible to move the body at will. The movement disorder is divided into complete paralysis, where movement is completely impossible, and incomplete paralysis, where movement is somewhat possible. The movement disorder refers to a state where voluntary movement of the body, such as movement of the limbs, trunk, neck, face, facial features, and tongue, is not possible voluntarily.

Meanwhile, the matters described as the background technology above are only intended to enhance understanding of the background of the present disclosure, and should not be taken as acknowledging that they correspond to prior art already known to those with ordinary skill in the art.

### DETAILED DESCRIPTION OF INVENTION

### Technical Problems

An object of the present disclosure is to provide an electronic device, a control method, and a computer program for evaluating and analyzing spiral drawing for diagnostic measurement of movement ability based on data such as writing pressure and speed acquired by drawing a line on a display surface.

Objects of the present disclosure are not limited to the problems mentioned above, and other objects not mentioned will be clearly understood by those skilled in the art from the description below.

### Technical Solution

In order to achieve the above-described objects, an electronic device for evaluating and analyzing spiral drawing for diagnostic measurement of movement ability according to one aspect of the present disclosure includes: a memory; a display that acquires a user command input to a surface and outputs an image generated according to the user command; and a processor connected to the memory and the display.

In this case, the processor tracks, through the display, a line generated by writing pressure to identify at least one stroke constituting the line, calculates a length of the stroke, calculates the writing pressure applied on a display with respect to the stroke, calculates a time taken to generate the stroke, and calculates a speed based on the length of the stroke and the time taken.

Here, the stroke may include at least two or more points and is equal to or less than a certain length or equal to or less than a certain angle.

Meanwhile, the processor may display, through the display, at least one of a pointer that receives the writing pressure initially, and a pointer line including the pointer, and a shape of the pointer line may include at least one of a straight line, a spiral, a circle, an oval, a square, a triangle, and an infinity shape.

In addition, the processor, when identifying at least one stroke constituting the line, in a case where the line is a spiral line, may identify a first point where the spiral line starts to be generated, may identify a second point that is most recently generated among a plurality of points forming the spiral line, and may divide a line between the first point and the second point into at least one stroke having an angle of 360 degrees or less in a spiral direction identified according to the tracking, thereby identifying at least one stroke constituting the spiral line, and when an angle of the stroke is 360 degrees, directions of vectors acquired for a starting point where the stroke is initially acquired and an ending point where the stroke is last acquired may be opposite to each other.

Additionally, the processor may acquire a pointer line for the spiral line as a template when the line is the spiral line, and acquire a normal line from the template toward the spiral line as a distance between the template and the spiral line.

Here, the distance may be acquired at 1 degree intervals with respect to the template.

Meanwhile, the processor may acquire the writing pressure applied on the display as a first value in steps of 0 to 4,096, convert the first value to a second value between 0 and 1, and calculate an average and standard deviation of the writing pressure applied on the display for the stroke.

Additionally, the processor, when the line is at least one of a spiral, a circle, an oval, a square, a triangle, and an infinity shape, may acquire a speed for the line, and acquire an average speed for each direction of the speed, and acquire points where the direction of the line changes at a predetermined angular interval.

In this case, the processor, when there are a plurality of strokes, may identify speed according to lengths of the strokes, identify the speed according to the order in which the strokes are acquired as the line is generated, calculate at least one first displacement, which is a speed change value according to the length of the stroke, calculate at least one second displacement, which is a speed change value according to the order of the strokes, and diagnose that a user who inputs a user command on the display needs improvement in movement ability when at least one of the first displacement and the second displacement exceeds a threshold value.

A method for controlling an electronic device according to one aspect of the present disclosure, includes: tracking generation of a line by writing pressure generated by a user input, by the electronic device; identifying at least one stroke constituting the line, by the electronic device; calculating a length of the stroke, by the electronic device; calculating the writing pressure applied on the display for the stroke, by the electronic device; calculating the time taken for the stroke to be generated, by the electronic device; and calculating a speed based on the length of the stroke and the time taken, by the electronic device.

At this time, the control method of the electronic device may further include providing at least one of a pointer that receives the writing pressure initially and a pointer line including the pointer, by the electronic device.

Additionally, the tracking of generation of a line may include acquiring a pointer line for the spiral line as a template when the line is the spiral line, and acquiring a normal line from the template toward the spiral line as a distance between the template and the spiral line, the distance being acquired at 1 degree intervals with respect to the template.

Meanwhile, the present disclosure further includes a computer program stored on a computer-readable recording medium, which is combined with a computer as hardware and enables the control method of an electronic device according to one aspect of the present disclosure to be performed.

Other specific details of the present disclosure are included in the detailed description and drawings.

### Effect of Invention

According to the electronic device, control method, and computer program for evaluating and analyzing spiral drawing for diagnostic measurement of movement ability of the present disclosure, diagnosis of movement ability can be performed without restrictions on location and expensive equipment.

In addition, since the time required for diagnosing the movement ability is short compared to the time required to manually complete a line, rapid diagnosis results can be acquired.

Effects of the present disclosure are not limited to the effects mentioned above, and other effects not mentioned will be clearly understood by those skilled in the art from the description below.

### BRIEF DESCRIPTION OF THE DRAWING

FIG. 1 is a configuration diagram of an electronic device according to one embodiment of the present disclosure.

FIG. 2 is a basic flow diagram according to one embodiment of the present disclosure.

FIGS. 3 to 6 are implementation diagrams according to one embodiment of the present disclosure.

### BEST MODE FOR CARRYING OUT THE INVENTION

The advantages and features of the present disclosure, and the methods for achieving them, will become clear with reference to the embodiments described below in detail with the accompanying drawings.

However, the present disclosure is not limited to the embodiments disclosed below, but can be implemented in various different forms, and these embodiments are provided only to make the disclosure of the present disclosure complete and to fully inform the scope of the present disclosure to those skilled in the art to which the present disclosure belongs, and the present disclosure is defined only by the scope of the claims.

The terminology used herein is for the purpose of describing embodiments and is not intended to limit the present disclosure.

In this specification, the singular also includes the plural unless specifically stated in the phrase. The terms “comprise” and/or “comprising” used in the specification do not exclude the presence or addition of one or more other components other than the mentioned components.

Throughout the specification, the same reference numerals refer to the same elements, and “and/or” includes each and every combination of the elements mentioned.

Although “first”, “second”, and the like are used to describe various elements, these elements are not limited by these terms.

These terms are only used to distinguish one element from another.

Therefore, it should be understood that a first element mentioned below may also be a second element within the technical concept of the present disclosure.

Unless otherwise defined, all terms (including technical and scientific terms) used in this specification may be used in a meaning that can be commonly understood by a person skilled in the art to which the present disclosure belongs.

In addition, terms defined in commonly used dictionaries are not to be ideally or excessively interpreted unless explicitly specifically defined.

The term “unit” or “module” used in the specification means a hardware component such as software, FPGA, or ASIC, and the “part” or “module” performs certain roles.

However, the “unit” or “module” is not limited to software or hardware. The “unit” or “module” may be configured to reside on an addressable storage medium and may be configured to cause one or more processors to execute.

Thus, as an example, a “unit” or “module” includes components such as software components, object-oriented software components, class components and task components, as well as processes, functions, attributes, procedures, subroutines, segments of program code, drivers, firmware, microcode, circuitry, data, databases, data structures, tables, arrays and variables.

The functionality provided within the components and “units” or “modules” may be combined into a smaller number of components and “units” or “modules” or further separated into additional components and “units” or “modules”.

Spatially relative terms such as “below,” “beneath,” “lower,” “above,” and “upper” may be used to easily describe the relationship of one component to other components as illustrated in the drawings.

Spatially relative terms should be understood to include different orientations of components when in use or operation in addition to the orientations depicted in the drawings.

For example, when components depicted in a drawing are flipped, a component described as “below” or “beneath” another component may end up “above” the other component.

Thus, the exemplary term “below” can include both the downward and upward directions. Components can also be oriented in other directions, and thus spatially relative terms can be interpreted according to orientation.

In this specification, a computer means any kind of hardware device including at least one processor, and may be understood to also encompass software configurations operating on the hardware device according to an embodiment.

For example, a computer may be understood to mean, but is not limited to, a smartphone, a tablet PC, a desktop, a laptop, and all user clients and applications running on each device.

Hereinafter, embodiments of the present disclosure will be described in detail with reference to the attached drawings.

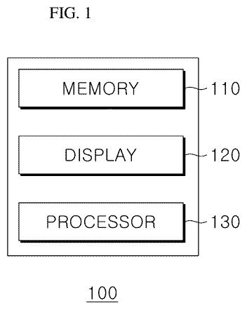

FIG. 1 is a block diagram of an electronic device according to one embodiment of the present disclosure.

An electronic device 100 for evaluating and analyzing spiral drawing for diagnostic measurement of movement ability according to one aspect of the present disclosure includes a memory 110, a display 120 for acquiring a user command input to a surface and outputting an image generated according to the user command, and a processor 130 connected to the memory 110 and the display 120.

In one embodiment, the electronic device 100 may include at least one of a server, a smartphone, a tablet personal computer, a mobile phone, a video phone, a laptop PC, a netbook computer, a notebook computer, a personal digital assistant (PDA), a portable multimedia player (PMP), and a wearable device.

Meanwhile, the memory 110 may store various programs and data necessary for the operation of the electronic device 100.

The memory 110 may be implemented as a non-volatile memory 110, a volatile memory 110, a flash memory 110, a hard disk drive HDD, or a solid-state drive SSD.

The processor 130 may control the overall operation of the electronic device 100 using various programs stored in the memory 110.

The processor 130 may include a RAM, a ROM, a graphic processing unit, a main CPU, first to n interfaces, and a bus. In this case, the RAM, the ROM, the graphic processing unit, the main CPU, the first to n interfaces, or the like may be connected to each other through a bus.

The RAM stores the O/S and application programs. Specifically, when the electronic device 100 is booted, the O/S is stored in the RAM, and various application data selected by the user can be stored in the RAM.

The ROM stores instruction sets for booting the system, or the like.

When a turn-on command is input and power is supplied, the main CPU copies the O/S stored in the memory 110 to the RAM according to the instruction stored in the ROM and executes the O/S to boot the system.

When booting is complete, the main CPU copies various application programs stored in the memory 110 to RAM and executes the application programs copied to RAM to perform various operations.

The graphic processing unit uses a calculation unit (not illustrated) and a rendering unit (not illustrated) to create a screen that includes various objects such as items, images, and text.

Here, the calculation unit may be configured to calculate attribute values, such as coordinate values, shape, size, and color, of each object to be displayed according to the layout of the screen using control commands received from the input unit.

Additionally, the rendering unit may be configured to generate screens with various layouts that include objects based on attribute values calculated in the calculation unit.

The screen generated in this rendering unit can be displayed within the display area of the display 120.

The main CPU accesses the memory 110 and performs booting using the OS stored in the memory 110.

Moreover, the main CPU performs various operations using various programs, contents, data, or the like stored in the memory 110.

The first to nth interfaces are connected to the various components described above. One of the first to nth interfaces may be a network interface that connects to an external device via a network.

Meanwhile, the processor 130 may include one or more cores (not illustrated) and a graphic processing unit (not illustrated) and/or a connection path (for example, a bus, or the like) for transmitting and receiving signals with other components.

The processor 130 according to one embodiment performs a method described in connection with the present disclosure by executing one or more instructions stored in a memory 110.

For example, the processor 130 may acquire new training data by executing one or more instructions stored in the memory 110, perform a test on the new acquired training data using a trained model, extract first training data in which labeled information is acquired with an accuracy equal to or more than a predetermined first reference value as a result of the test, delete the extracted first training data from the new training data, and re-train the trained model using the new training data from which the extracted training data has been deleted.

Meanwhile, the processor 130 may further include a random access memory (RAM) (not illustrated) and a read-only memory (ROM) (not illustrated) that temporarily and/or permanently store signals (or data) processed within the processor 130.

Additionally, the processor 130 may be implemented in the form of a system on chip (SoC) including at least one of a graphic processing unit, RAM, and ROM.

The memory 110 may store programs (one or more instructions) for processing and controlling the processor 130.

Programs stored in the memory 110 may be divided into a plurality of modules according to function.

The display 120 is configured to visually output various information.

The display 120 may be implemented as a liquid crystal display (LCD), a plasma display panel (PDP), an organic light emitting diode (OLED), a transparent OLED (TOLED), a micro-LED, or the like, but is not limited thereto and may include various types of displays known in the art.

The display 120 may be implemented in the form of a touch screen capable of detecting a user's touch operation, and may also be implemented as a flexible display capable of being folded or bent.

Meanwhile, the electronic device 100 further includes a communication unit, and may obtain a user command input from the terminal to the display surface of the terminal in real time.

The communication unit may communicate with external devices.

In particular, the communication unit may include various communication chips such as a Wi-Fi chip, a Bluetooth chip, a wireless communication chip, an NFC chip, a low-power Bluetooth chip BLE chip, or the like.

In this case, the WiFi chip, Bluetooth chip, and NFC chip communicate in the LAN mode, WiFi mode, Bluetooth mode, and NFC mode, respectively.

When using the Wi-Fi chip or Bluetooth chip, various connection information such as SSID and session key is first sent and received, and then communication is established using this information, after which various information may be sent and received.

A wireless communication chip refers to a chip that performs communication according to various communication standards such as IEEE, Zigbee, 3rd Generation (3G), 3rd Generation Partnership Project (3GPP), Long Term Evolution (LTE), and 5th Generation (5G).

The terminal includes a communication unit for communicating with the electronic device 100, a display for acquiring a user command input to a surface, and a processor connected to the communication unit and the display.

In one embodiment, the terminal may include at least one of a smartphone, a tablet personal computer, a mobile phone, a video phone, a laptop PC, a netbook computer, a notebook computer, a personal digital assistant PDA, a portable multimedia player PMP, and a wearable device.

The processor performs the overall operations of the terminal.

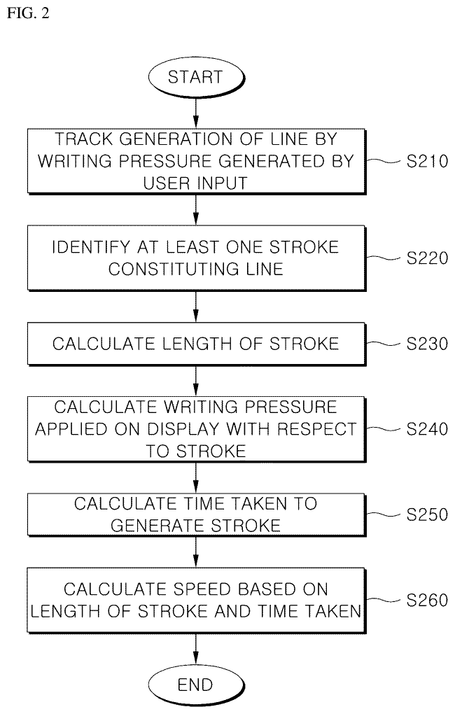

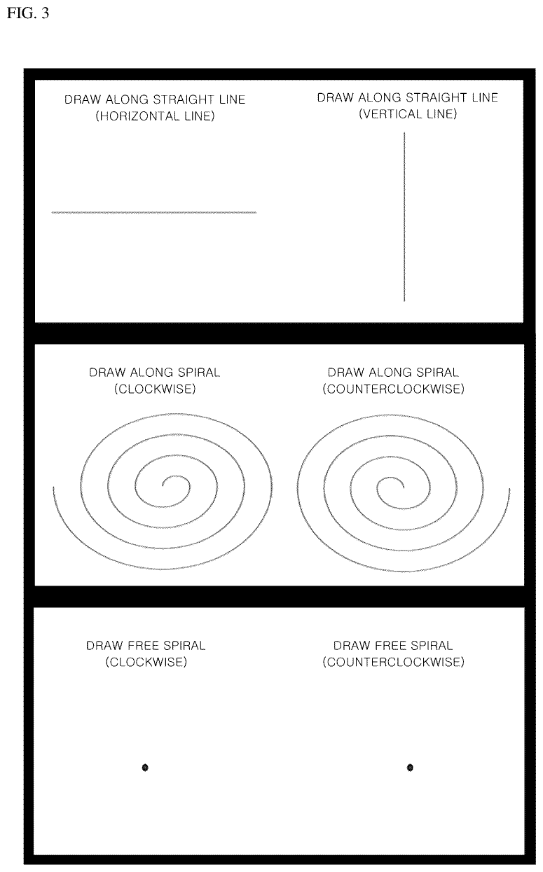

FIG. 2 is a basic flowchart according to one embodiment of the present disclosure, and FIGS. 3 to 6 are implementation diagrams according to one embodiment of the present disclosure.

As illustrated in FIG. 2, the electronic device 100 for evaluating and analyzing spiral drawing for diagnostic measurement of movement ability according to one aspect of the present disclosure tracks the generation of a line by writing pressure through the display 120 (S210).

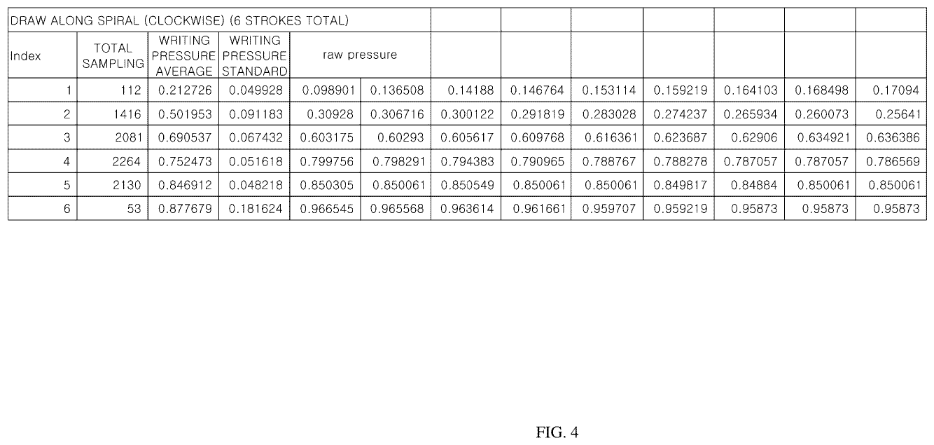

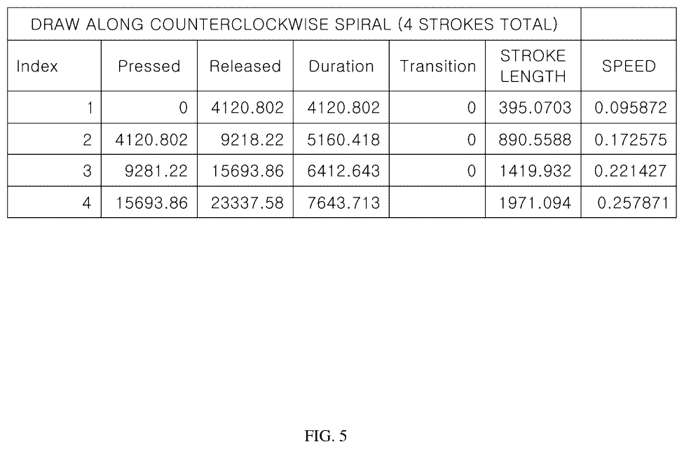

Accordingly, the electronic device 100 identifies at least one stroke constituting the line (S220), calculates the length of the stroke (S230), calculates the writing pressure applied on the display 120 with respect to the stroke as illustrated in FIG. 4 (S240), calculates a time taken to generate the stroke (S250), and calculates speed based on the length of the stroke and the time taken as illustrated in FIG. 5 (S260).

In this case, the stroke includes at least two points, is equal to or less than a certain length or equal to or less than a certain angle.

For example, for a line consisting of 80 cm in length, when strokes are separated at 7 cm intervals, a total of 12 strokes are identified by the electronic device 100, and the last generated stroke has a length of 3 cm.

In addition, for a spiral line that is rotated twice and drawn at an additional 30 degrees, when the strokes are divided into 360-degree angles, a total of three strokes are identified by the electronic device 100, and for the last generated stroke, an angle between a first line formed by the starting point of the stroke and the first point, which is the starting point of the line, and a second line formed by the last point of the stroke and the first point is 30 degrees.

In this case, one rotation is made so that the direction at the starting point and the direction at the end point of the rotation are opposite to each other in the direction in which the line is drawn by the rotation.

Meanwhile, as illustrated in FIG. 3, the electronic device 100 displays at least one of a pointer point (free drawing) that receives pressure input initially and a pointer line (draw along) that includes the pointer point through the display 120, so that the user can input a line on the surface of the display 120 according to what is indicated by at least one of the pointer point and the pointer line.

In this case, the shape of the pointer line may include at least one of a straight line, a spiral, a circle, an oval, a square, a triangle, and an infinity shape.

Meanwhile, when performing Step S220, when the line is a spiral line, the electronic device 100 identifies a first point where the spiral line starts to be generated, and identifies a second point that is most recently generated among a plurality of points forming the spiral line.

Accordingly, the electronic device 100 can identify at least one stroke constituting the spiral line by dividing a line between the first point and the second point into at least one stroke having an angle of 360 degrees or less in a spiral direction identified according to the tracking.

In this case, when an angle of the stroke is 360 degrees, directions of vectors acquired for a starting point where the stroke is initially acquired and an ending point where the stroke is last acquired are opposite to each other.

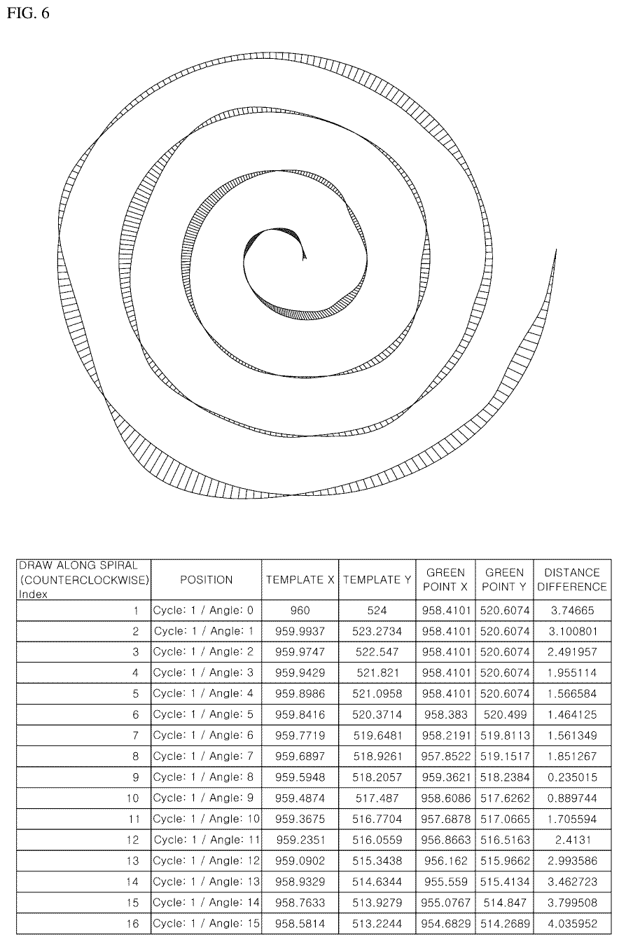

In addition, when the line is a spiral, as illustrated in FIG. 6, the electronic device 100 acquires a pointer line for the spiral line as a template, and acquires a normal line from the template toward the spiral line as the distance between the template and the spiral line.

In this case, the distance is acquired at 1 degree intervals with respect to the template.

For example, a total of 360 distances are acquired by the electronic device 100 in 1-degree angular intervals from 1 degree to 360 degrees for one stroke, and for only the stroke initially acquired with respect to a spiral line, a distance is acquired even at 0 degrees, so a total of 361 distances are acquired by the electronic device 100 for the initially acquired stroke.

As an example, the electronic device 100 calculates the ratio of the acquired distance to the length of the stroke, and when the ratio exceeds a preset reference ratio by a threshold amount or more, the electronic device diagnoses that the user who input the user command on the display 120 needs improvement in the movement ability.

Meanwhile, in performing Step S240, the electronic device 100 may obtain the writing pressure applied on the display 120 as a first value in steps of 0 to 4,096, and, as illustrated in FIG. 4, convert the first value to a second value between 0 to 1, and calculate an average of the writing pressure applied on the display 120 for the stroke and a first standard deviation for the average.

In an embodiment, the electronic device 100 identifies a second standard deviation matching the average of the input writing pressure stroke by stroke based on an indicator previously stored in the memory 110 for the standard deviation of the average of the writing pressure converted to a second value between 0 and 1, and when there is a standard deviation exceeding the matching second standard deviation among the first standard deviations acquired stroke by stroke, calculates a ratio (for example, 100*first standard deviation/second standard deviation) at which the first standard deviation exceeds the matching second standard deviation, and when the calculated ratio exceeds a threshold ratio, the electronic device may diagnose that the user who input the user command on the display 120 needs improvement in the movement ability.

In performing Step S260, when the line is at least one of a spiral, a circle, an oval, a square, a triangle, and an infinity shape, that is, when the line includes at least one curved stroke, or the line forms a closed shape, the electronic device 100 may obtain a speed for the line and obtain an average speed for each direction of the speed, and obtain points where the direction changes for the line at constant angular intervals.

In this case, when there are a plurality of strokes, the electronic device 100 identifies the speed according to the length of the strokes, and identifies the speed according to the order in which the strokes are acquired as the line is generated.

Accordingly, the electronic device 100 may calculate at least one first displacement, which is a speed change value according to the length of the stroke, and calculate at least one second displacement, which is a speed change value according to the order of the strokes.

When at least one of the calculated at least one first displacement and at least one second displacement exceeds the threshold value, the electronic device 100 may diagnose that the user who input the user command on the display 120 needs improvement in the movement ability.

For example, the first displacement is a value of the speed difference between two adjacent strokes calculated by the electronic device 100 by listing the plurality of strokes in increasing order of length, starting from the shortest stroke to the longest stroke.

The second displacement is calculated by the electronic device 100 by listing the plurality of strokes in the order in which the strokes are acquired, and calculating the speed difference value between two adjacent strokes.

In an embodiment, when at least one first displacement acquired exceeds the threshold value, the electronic device 100 may identify at least one second stroke that is adjacent in sequence generated for two first strokes that are adjacent in sequence matching the first displacement that exceeds the threshold value.

Accordingly, the electronic device 100 calculates the average speed of the first stroke and the second stroke, calculates a difference value that each of the first stroke and the second stroke has with respect to the average speed of the calculated first stroke and the second stroke, calculates the ratio that the calculated difference value has with respect to the average speed, and only when the calculated ratio exceeds a certain ratio, diagnoses that the user who input the user command on the display 120 needs improvement in the movement ability.

Even when the first displacement exceeds the threshold, by considering the change in the direction in which the user's joints move and the change in the distance the hand has from the body according to the generated order, the electronic device 100 can determine that the difference value in the average speed between neighboring strokes is a difference value that can occur with respect to the average speed, and that this is caused by the change in the direction in which the user's joints move and the change in the distance the hand has from the body, rather than by low movement ability.

In this case, when the number of first displacements exceeding the threshold value is a certain number or more, the electronic device 100 may diagnose that the user who input the user command on the display 120 needs improvement in the movement ability for the joint.

In a control method of an electronic device 100 according to one aspect of the present disclosure, the method may include a step of tracking generation of a line by writing pressure generated by a user input, by the electronic device 100, a step of identifying at least one stroke constituting the line, by the electronic device 100, a step of calculating a length of the stroke, by the electronic device 100, a step of calculating the writing pressure applied on the display for the stroke, by the electronic device 100, a step of calculating the time taken for the stroke to be generated, by the electronic device 100, and a step of calculating a speed based on the length of the stroke and the time taken, by the electronic device 100.

In this case, the control method of the electronic device 100 may include a step of providing at least one of a pointing point that receives the writing pressure initially and a pointing line including the pointing point, by the electronic device 100.

Additionally, the step of tracking the generation of the line may include a step of acquiring a pointer line for the spiral line as a template when the line is the spiral line, and a step of acquiring a normal line from the template toward the spiral line as a distance between the template and the spiral line, the distance being acquired at 1 degree intervals with respect to the template.

Meanwhile, the present disclosure includes a computer program stored on a computer-readable recording medium which is combined with a computer as hardware and enables the control method of the electronic device 100 according to one aspect of the present disclosure to be performed.

The steps of a method or algorithm described in connection with the embodiments of the present disclosure may be implemented directly in hardware, implemented in a software module executed by hardware, or implemented by a combination of these. The software module may reside in a random access memory (RAM), a read only memory (ROM), an erasable programmable ROM (EPROM), an electrically erasable programmable ROM (EEPROM,) a flash memory, a hard disk, a removable disk, a CD-ROM, or any other form of computer-readable recording medium well known in the art to which the present disclosure pertains.

Additionally, different embodiments of the present disclosure may be complementary or combined with each other.

The components of the present disclosure can be implemented as a program (or application) to be executed in combination with a computer, which is hardware, and stored in a medium.

Components of the present disclosure may be implemented as software programming or software elements, and similarly, embodiments may be implemented in a programming or scripting language, such as C, C++, Java, assembler, Python, or the like, including various algorithms implemented as combinations of data structures, processes, routines, or other programming constructs.

Functional aspects may be implemented as algorithms running on one or more processors.

Although the embodiments of the present disclosure have been described above with reference to the attached drawings, those skilled in the art will appreciate that the present disclosure can be implemented in other specific forms without changing the technical idea or essential features thereof.

Therefore, it should be understood that the embodiments described above are exemplary in all respects and not restrictive.

### MODE FOR CARRYING OUT THE INVENTION

In order to achieve the above-described objects, an electronic device for evaluating and analyzing spiral drawing for diagnostic measurement of movement ability according to one aspect of the present disclosure includes: a memory, a display that acquires a user command input to a surface and outputs an image generated according to the user command, and a processor connected to the memory and the display.

In this case, the processor tracks, through the display, a line generated by writing pressure to identify at least one stroke constituting the line, calculates a length of the stroke, calculates the writing pressure applied on a display with respect to the stroke, calculates a time taken to generate the stroke, and calculates a speed based on the length of the stroke and the time taken.

Here, the stroke may include at least two or more points and is equal to or less than a certain length or equal to or less than a certain angle.

Meanwhile, the processor may display, through the display, at least one of a pointer that receives the writing pressure initially, and a pointer line including the pointer, and a shape of the pointer line may include at least one of a straight line, a spiral, a circle, an oval, a square, a triangle, and an infinity shape.

In addition, the processor, when identifying at least one stroke constituting the line, in a case where the line is a spiral line, may identify a first point where the spiral line starts to be generated, may identify a second point that is most recently generated among a plurality of points forming the spiral line, and may divide a line between the first point and the second point into at least one stroke having an angle of 360 degrees or less in a spiral direction identified according to the tracking, thereby identifying at least one stroke constituting the spiral line, and when an angle of the stroke is 360 degrees, directions of vectors acquired for a starting point where the stroke is initially acquired and an ending point where the stroke is last acquired may be opposite to each other.

Additionally, the processor may acquire a pointer line for the spiral line as a template when the line is the spiral line, and acquire a normal line from the template toward the spiral line as a distance between the template and the spiral line.

Here, the distance may be acquired at 1 degree intervals with respect to the template.

Meanwhile, the processor may acquire the writing pressure applied on the display as a first value in steps of 0 to 4,096, convert the first value to a second value between 0 and 1, and calculate an average and standard deviation of the writing pressure applied on the display for the stroke.

Additionally, the processor, when the line is at least one of a spiral, a circle, an oval, a square, a triangle, and an infinity shape, may acquire a speed for the line, and acquire an average speed for each direction of the speed, and acquire points where the direction of the line changes at a predetermined angular interval.

In this case, the processor, when there are a plurality of strokes, may identify speed according to lengths of the strokes, identify the speed according to the order in which the strokes are acquired as the line is generated, calculate at least one first displacement, which is a speed change value according to the length of the stroke, calculate at least one second displacement, which is a speed change value according to the order of the strokes, and diagnose that a user who inputs a user command on the display needs improvement in movement ability when at least one of the first displacement and the second displacement exceeds a threshold value.

A method for controlling an electronic device according to one aspect of the present disclosure, includes tracking generation of a line by writing pressure generated by a user input, by the electronic device, identifying at least one stroke constituting the line, by the electronic device; calculating a length of the stroke, by the electronic device, calculating the writing pressure applied on the display for the stroke, by the electronic device, calculating the time taken for the stroke to be generated, by the electronic device; and calculating a speed based on the length of the stroke and the time taken, by the electronic device.

At this time, the control method of the electronic device may further include providing at least one of a pointer that receives the writing pressure initially and a pointer line including the pointer, by the electronic device.

Additionally, the tracking of generation of a line may include acquiring a pointer line for the spiral line as a template when the line is the spiral line, and acquiring a normal line from the template toward the spiral line as a distance between the template and the spiral line, the distance being acquired at 1 degree intervals with respect to the template.

Meanwhile, the present disclosure further includes a computer program stored on a computer-readable recording medium, which is combined with a computer as hardware and enables the control method of an electronic device according to one aspect of the present disclosure to be performed.

1. An electronic device, comprising:
a memory;
a display that acquires a user command input to a surface and outputs an image generated according to the user command; and
a processor connected to the memory and the display,
wherein the processor
tracks, through the display, a line generated by writing pressure to identify at least one stroke constituting the line
calculates a length of the stroke,
calculates the writing pressure applied on a display with respect to the stroke,
calculates a time taken to generate the stroke, and
calculates a speed based on the length of the stroke and the time taken, and
the stroke includes at least two or more points and is equal to or less than a certain length or equal to or less than a certain angle.

2. The electronic device of claim 1, wherein the processor displays, through the display, at least one of a pointer that receives the writing pressure initially, and a pointer line including the pointer, and
a shape of the pointer line includes at least one of a straight line, a spiral, a circle, an oval, a square, a triangle, and an infinity shape.

3. The electronic device of claim 2, wherein the processor,
when identifying at least one stroke constituting the line, in a case where the line is a spiral line, identifies a first point where the spiral line starts to be generated,
identifies a second point that is most recently generated among a plurality of points forming the spiral line, and
divides a line between the first point and the second point into at least one stroke having an angle of 360 degrees or less in a spiral direction identified according to the tracking, thereby identifying at least one stroke constituting the spiral line, and
when an angle of the stroke is 360 degrees, directions of vectors acquired for a starting point where the stroke is initially acquired and an ending point where the stroke is last acquired are opposite to each other.

4. The electronic device of claim 3, wherein the processor,
acquires a pointer line for the spiral line as a template when the line is the spiral line, and
acquires a normal line from the template toward the spiral line as a distance between the template and the spiral line, the distance being acquired at 1 degree intervals with respect to the template.

5. The electronic device of claim 1, wherein the processor,
acquires the writing pressure applied on the display as a first value in steps of 0 to 4,096,
converts the first value to a second value between 0 and 1, and
calculates an average and standard deviation of the writing pressure applied on the display for the stroke.

6. The electronic device of claim 5, wherein the processor,
when the line is at least one of a spiral, a circle, an oval, a square, a triangle, and an infinity shape, acquires a speed for the line, and
acquires an average speed for each direction of the speed, and acquires points where the direction of the line changes at a predetermined angular interval.

7. The electronic device of claim 1, wherein the processor,
when there are a plurality of strokes, identifies speed according to lengths of the strokes,
identifies the speed according to the order in which the strokes are acquired as the line is generated,
calculates at least one first displacement, which is a speed change value according to the length of the stroke,
calculates at least one second displacement, which is a speed change value according to the order of the strokes, and
diagnoses that a user who inputs a user command on the display needs improvement in movement ability when at least one of the first displacement and the second displacement exceeds a threshold value.

8. A method for controlling an electronic device according to claim 1, the method comprising:
tracking generation of a line by writing pressure generated by a user input, by the electronic device;
identifying at least one stroke constituting the line, by the electronic device;
calculating a length of the stroke, by the electronic device;
calculating the writing pressure applied on the display for the stroke, by the electronic device;
calculating the time taken for the stroke to be generated, by the electronic device; and
calculating a speed based on the length of the stroke and the time taken, by the electronic device.

9. The method of claim 8, further comprising providing at least one of a pointer that receives the writing pressure initially and a pointer line including the pointer, by the electronic device.

10. The method of claim 9, wherein the tracking of generation of a line includes
acquiring a pointer line for the spiral line as a template when the line is the spiral line, and
acquiring a normal line from the template toward the spiral line as a distance between the template and the spiral line, the distance being acquired at 1 degree intervals with respect to the template.

11. A computer program stored on a computer-readable recording medium, which is combined with a computer as hardware and enables the method of claim 8 to be performed.
